(19) United States
(12) Patent Application Publication
(10) Pub. No.: US 2023/0300949 A1
(43) Pub. Date: Sep. 21, 2023

(54) APPARATUSES AND METHODS FOR SEQUENTIAL HEATING OF CRYO-FLUID IN CRYOABLATION SYSTEMS

(71) Applicant: Varian Medical Systems, Inc., Palo Alto, CA (US)

(72) Inventor: Hongxuan Zhang, Austin, TX (US)

(21) Appl. No.: 17/655,204

(22) Filed: Mar. 17, 2022

(51) Int. Cl. H05B 1/02; G05D 23/19; A61B 18/02

(52) U.S. Cl. CPC H05B 1/0244; A61B 18/02; G05D 23/1917; H05B 1/025; A61B 2018/00577

(57) ABSTRACT

A system for performing a cryoablation treatment may include at least one computing device configured to obtain temperature information at a plurality of heating locations on a cryo-fluid supply. The plurality of heating locations includes a first heating location and a second heating location. The computing device is also configured to compare a first temperature at the first heating location to an expected first temperature and to initiate a first heating cycle at the first heating location if the first temperature at the first heating location is less than the expected first temperature. The computing device also compares a second temperature at the second heating location to an expected second temperature wherein the second heating location disposed downstream of the first heating location and initiates a second heating cycle at the second heating location if the second temperature is less than the expected second temperature.

### FIELD

The present disclosure relates to apparatuses and methods for the sequential heating of a cryo-fluid in a cryoablation system.

### BACKGROUND

This section provides background information related to the present disclosure which is not necessarily prior art.

Systems and methods for providing cryoablation treatments may include cryoablation probes that are introduced at or near target tissue in a patient. A cryoablation system may include an extremely cold cryo-fluid (liquid, gas, or mixed phase) that may be passed through a probe in thermal contact with the target tissue. Heat from the tissue passes from the tissue, through the probe, and into the fluid that removes heat from the targeted tissue. This removal of heat causes tissue to freeze, resulting in the destruction of the targeted tissue. The cryo-fluid may also be heated subsequent to the freezing cycle. The heating may thaw the frozen tissue to allow the cryoprobe to be removed from the tissue. The heating may also be used to coagulate blood in instances of bleeding at the cryoablation site.

Traditional or existing systems and methods often rely on predetermined treatment procedures that determine system settings and cycle parameters through testing in a laboratory setting. Such predetermined treatment procedures often do not account for differences between patients or for circumstances that may arise in the course of a cryoablation treatment. Furthermore, traditional or existing systems and methods often require power-intensive heating processes that are inefficient and costly. Still further, the traditional or existing systems may result in undesirable damage to healthy tissue that may be located in areas near to or surrounding the target tissue. Therefore, improvements are needed to improve the efficiency and efficacy of heating processes in cryoablation systems. Such improvements can also reduce the likelihood of damage to healthy tissue in patients and allow cryoablation treatments to better account for differences between patients and to adapt to varying circumstances that may arise during the course of treatment.

### SUMMARY

This section provides a general summary of the disclosure, and is not a comprehensive disclosure of its full scope or all of its features.

In some embodiments of the present disclosure a cryoablation system is provided that may actively and continuously monitor and adjust a plurality of heaters positioned at a plurality of locations on a cryo-fluid supply. The system may include a computing device that can receive temperature information from each of the plurality of heating locations and sequentially initiating heating cycles at each heating location. The computing device may also adjust a heating profile of a heater located at each of the heating locations in response to the temperature information received from each heating location.

In some embodiments of the present disclosure, a system for performing a cryoablation treatment is provided. The system may include at least one computing device configured to obtain temperature information at a plurality of heating locations on a cryo-fluid supply. The plurality of heating locations may include a first heating location and a second heating location. The computing device may also be configured to compare a first temperature at the first heating location to an expected first temperature and to initiate a first heating cycle at the first heating location if the first temperature at the first heating location is less than the expected first temperature. The computing device may also be configured to compare a second temperature at the second heating location to an expected second temperature wherein the second heating location is disposed downstream of the first heating location and to initiate a second heating cycle at the second heating location if the second temperature is less than the expected second temperature.

In one aspect, the cryoablation system may include one or more temperature sensors coupled to the at least one computing device, wherein the at least one computing device obtains the temperature information from one or more temperature sensors.

In another aspect, the cryoablation system may include a plurality of heating coils each positioned at a corresponding heating location of the plurality of heating locations.

In another aspect, each heating coil is configured to send a temperature signal to the at least one computing device, the temperature signal corresponding to a temperature at the corresponding location on the cryo-fluid supply.

In another aspect, each heating coil is also configured to selectively heat a cryo-fluid in the cryo-fluid supply at the corresponding heating location.

In another aspect, the computing device is configured to energize a first heater at the first heating location to initiate the first heating cycle.

In another aspect, the at least one computing device if further configured to compare a third temperature at a third heating location to an expected third temperature wherein the third heating location is disposed downstream of the first heating location and the second heating location and to initiate a third heating cycle at the third heating location if the third temperature is less than the expected third temperature.

In another aspect, the second heating location is disposed upstream of a cryoprobe.

In another aspect, the first heating cycle may include a first temperature profile and the second heating cycle comprises a second heating profile wherein the first temperature profile and the second temperature profile are different.

In another aspect, the first heating cycle may include an amplitude modulated power profile.

In another aspect, the first heating cycle may include a pulse width modulated (PWM) power profile.

In some embodiments of the present disclosure, a method of sequentially heating cryo-fluid is provided. The method may include obtaining temperature information at a plurality of heating locations on a cryo-fluid supply wherein the plurality of heating locations includes a first heating location and a second heating location. The method may also include comparing a first temperature at the first heating location to an expected first temperature and initiating a first heating cycle at the first heating location if the first temperature at the first heating location is less than the expected first temperature. The method may also include comparing a second temperature at the second heating location to an expected second temperature wherein the second heating location is disposed downstream of the first heating location and initiating a second heating cycle at the second heating location if the second temperature is less than the expected second temperature.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic illustrating an example cryoablation system in accordance with some embodiments of the present disclosure.

FIG. 2 is a diagram illustrating an example sequential heating profile in accordance with some embodiments of the present disclosure.

FIG. 3 is a schematic illustrating an example heater and example heating methods in accordance with some embodiments of the present disclosure.

FIG. 4 is a flow chart illustrating an example method of sequential heating in accordance with some embodiments of the present disclosure.

FIG. 5 is a flow chart illustrating another example method of sequential heating in accordance with some embodiments of the present disclosure.

FIG. 6 is a flow chart illustrating another example method of sequential heating in accordance with some embodiments of the present disclosure.

FIG. 7 is a diagram illustrating an example computing device that may be used in connection with one or more methods of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature’s relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

In accordance with some embodiments of the present disclosure, a cryoablation apparatus or system is provided that is configured to allow a thaw or coagulation cycle to be controlled monitored and/or optimized to improve the performance over traditional or existing systems and methods. The cryoablation systems and methods of the present disclosure can collect, store and process real-time information regarding one or more temperatures or other conditions of the cryoablation system. The cryoablation systems and methods of the present disclosure may also heat one or more portions, regions according to a predetermined profile. The predetermined heating profile may provide for the sequential heating of different regions of the cryoablation system. Such predetermined heating profiles can improve the performance of the thaw and/or coagulation profiles to improve efficiency, reduce cost, reduce the likelihood of damage to healthy tissues and/or improve the efficacy of the cryoablation treatment over traditional or existing methods and systems.

The cryoablation systems of the present disclosure may also use one or more elements or methods as described in U.S. Pat. Application No. TBD entitled “APPARATUSES AND METHODS FOR ADAPTIVELY CONTROLLING CRYOABLATION SYSTEMS” filed on the same day as the present application by Varian Medical Systems, Inc., U.S. Pat. Application No. TBD entitled “APPARATUSES AND METHODS FOR MONITORING AND CONTROLLING BLEEDING DURING CRYOABLATION TREATMENTS” filed on the same day as the present application by Varian Medical Systems, Inc., and U.S. Pat Application No. TBD entitled “APPARATUSES AND METHODS FOR THE CONTROL AND OPTIMIZATION OF ICE FORMATION DURING CYROABLATION TREATMENTS” filed on the same day as the present application by Varian Medical Systems, Inc., the disclosures of which are hereby incorporated by reference in their entireties.

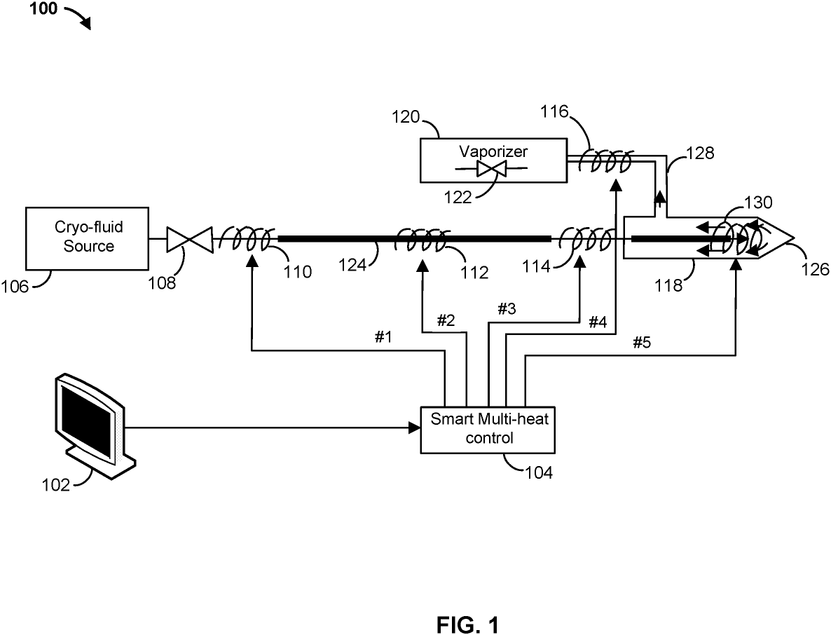

Turning now to FIG. 1, an example cryoablation system 100 is shown. The cryoablation system 100 may include a cryoablation computing device 102, a smart multi-heat control 104, a cryo-fluid source 106, an inlet valve 108, a first heater 110, a second heater 112, a third heater 114, a fourth heater 116, a cryoprobe 118, a vaporizer 120, an exhaust valve 122, and a cryo-fluid supply 124. The cryo-fluid source 106, the inlet valve 108, the first heater 110, the second heater 112, the third heater 114, the fourth heater 116, the cryoprobe 118, the vaporizer 120, the exhaust valve 122, and the cryo-fluid supply 124 may operate to deliver a cryo-fluid from the cryo-fluid source 106 to the cryoprobe 118 to perform a cryoablation treatment. The cryo-fluid (e.g., liquid nitrogen) can be stored in the cryo-fluid source 106, such as a dewar or other suitable container, and then delivered to the cryoprobe 188 via the cryo-fluid supply 124. The cryo-fluid may expand at a tip 126 of the cryoprobe 118 and cool the tip 126 of the cryoprobe 118 to a temperature at which the tissue of a patient surrounding the cryoprobe 118 begins to freeze forming an iceball.

The cryoprobe 118 can be positioned at or near a target tissue (e.g., a tumor) in the patient. In this manner, the target tissue can be frozen destroying the target tissue. Once a freezing cycle is complete, a thaw cycle can be initiated. The thaw cycle can be used so that the cryoprobe 118 can be extracted. The thaw cycle can also stop the iceball from continuing to form and/or prevent damage to healthy tissues that may located near to or surround the target tissue that is being frozen or is near the iceball. In still other examples, the thaw cycle may be used to coagulate or otherwise reduce or stop bleeding that may occur at or near the cryoablation site. The cryoprobe 118 may be heated to a temperature that causes the coagulation of blood to occur (e.g., at or above 100° F. or at or above 110° F.). During a thaw cycle, a probe heater 130 may be used to heat the cryo-fluid and/or the cryoprobe 118. The cryo-fluid may be evacuated from the cryoprobe 118 to allow the cryoprobe to warm. The cryo-fluid may flow from the cryoprobe 118 through a return line 128 and be vaporized by the vaporizer 120 and/or exhausted to the environment via the exhaust valve 122. During various cryoablation treatments, one or more freezing and/or thaw cycles may be used.

A treatment plan can be determined prior to the performance of the cryoablation treatment. The treatment plan can detail and/or describe the various steps of the process and various aspects of the treatment such as the types of equipment to be used, a positioning of the cryoprobe, temperatures of the cryoprobe, duration of freezing and thaw cycles as well as a quantity of cycles. The treatment plan may be determined by a medical professional and/or by others. In some examples, the cryoablation computing device 102 may determine or recommend a treatment plan after health, patient, and other information is input into the cryoablation computing device 102 or is retrieved or otherwise obtained by the cryoablation computing device 102.

In traditional or existing systems and methods, the thaw cycle may be performed by heating the cryoprobe using the probe heater 130 that is located in or at the tip 126 of the cryoprobe 118. The use of single heater and/or use of a heater at only the tip 126 of the cryoprobe 118 can result in several disadvantages. First, the probe heater 130, if energized at elevated levels to stop the formation of the iceball, may require significant amounts of power to provide sufficient levels of heating. Such power levels can exceed 20 W or more. Also, the probe heater 130 may heat quickly and without sufficient control such that the probe may burn or otherwise damage healthy tissue that may be located proximate to the cryoprobe 118.

The systems and method provide improvements over traditional and existing systems. The cryoablation system 100, for example, may provide a closed loop information system that provides information regarding temperatures at various locations along the cryo-fluid supply 124 and/or the cryoprobe 118. This information can be used to continuously monitor and control the heating that is being applied to the system. In addition, the heating may be applied by heaters located at two or more locations along the cryo-fluid supply 124. Such a process may allow the temperature and thaw cycle of the cryoablation system 100 to be accurately and precisely controlled. In addition, the multiple heating locations and the temperature and power profiles applied to such heating locations can allow the thaw cycles to be performed at lower levels of power (e.g., less 5 W and in some examples at or below 2 W).

As shown in FIG. 1, the cryoablation computing device 102 may be coupled to the smart multi-heat control 104. The first heater 110, the second heater 112, the third heater 114, the fourth heater 116, and the cryoprobe heater 130 may be coupled to the smart multi-heat control 104. The smart multi-heat control 104 can operate to independently energize and/or activate each of the first heater 110, the second heater 112, the third heater 114, the fourth heater 116, and the cryoprobe heater 130. The first heater 110, the second heater 112, the third heater 114, the fourth heater 116, and the cryoprobe heater 130 may also be configured to provide a temperature measurement of the temperature locations associated with each of the first heater 110, the second heater 112, the third heater 114, the fourth heater 116, and the cryoprobe heater 130. The smart multi-heat control 104 can operate to transfer the temperature information to the cryoablation computing device 102. In addition, the smart multi-heat control 104 can receive instructions from the cryoablation computing device 102 regarding the heating durations, heating times, and power levels to be delivered to each of the first heater 110, the second heater 112, the third heater 114, the fourth heater 116, and the cryoprobe heater 130.

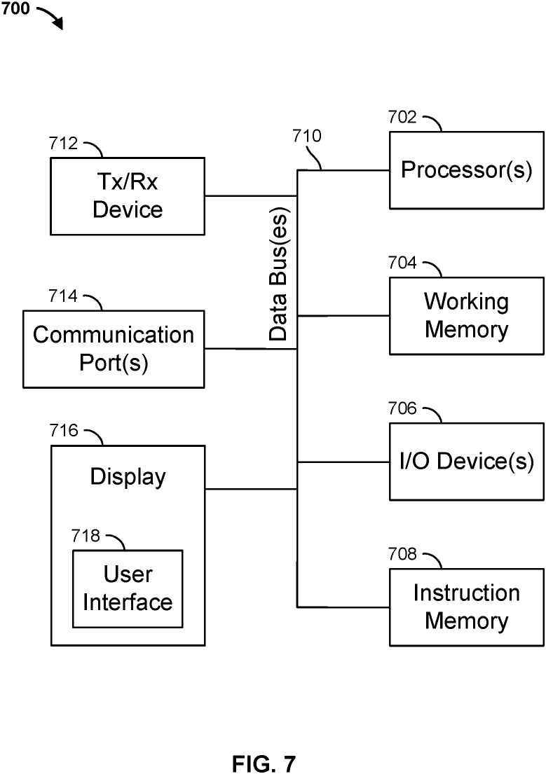

The cryoablation computing device 102 can be any suitable computing device (such as that described in FIG. 7) that can obtain data and information, process such information and deliver instructions to the smart multi-heat control 104. In some examples, the cryoablation computing device 102 may be a workstation, server, laptop, tablet or other suitable computing device. The smart multi-heat control 104 can be a suitable controller such as a programmable logic controller, data acquisition and control unit or the like.

Each of first heater 110, the second heater 112, the third heater 114, the fourth heater 116, and the cryoprobe heater 130 may be configured as any suitable heating device to perform the methods described herein. In one example, each of the first heater 110, the second heater 112, the third heater 114, the fourth heater 116, and the cryoprobe heater 130 can be configured as a heater coil made of suitable heating coil wire that not only heats when energized by a power source but also can provide a temperature measurement such as by an impedance measurement of the heater coil.

In some examples, the treatment plan for a cryoablation procedure can be obtained by the cryoablation computing device 102 The cryoablation computing device 102 may determine recommended freezing and thaw cycle settings and profiles that can be used during the procedure to obtain the desired results such as iceball size, duration and the like. The cryoablation computing device 102 may also determine recommended thaw profiles that can be used to thaw the cryoprobe 118 and the surrounding tissues in between freezing cycles. Such thaw cycles can also recommend operational settings for the coagulation of bleeding that may occur.

The recommended thaw cycle profiles that may be determined by the cryoablation computing device 102 and/or implemented by the cryoablation computing device 102 may include settings for a duration that each heater is energized and the power levels used at each heater. In some examples, the heaters may be energized at different times and in a sequential process so as to reduce the amount of power needed to raise the temperature of the cryo-fluid to a desired a level. Such a process can also allow the temperature of the cryo-fluid to be adaptively and accurately controlled during the cryoablation procedure.

In some examples, the first heater 110, the second heater 112, the third heater 114, the fourth heater 116, and the cryoprobe heater 130 may not be energized at the same time. In some examples, the first heater 110, the second heater 112, the third heater 114, the fourth heater 116, and the cryoprobe heater 130 are energized in a sequential manner starting with a heater located at an upstream position and then each heater is sequentially heated (if necessary) in a downstream sequence. It should be appreciated that the cryoablation system 100 include four heaters and a cryoprobe heater. In other examples, the cryoablation system 100 may include other quantities or other arrangement of heaters. In some examples, the cryoablation system 100 includes more than four heaters. In other examples, the cryoablation system 100 may include less than four heaters.

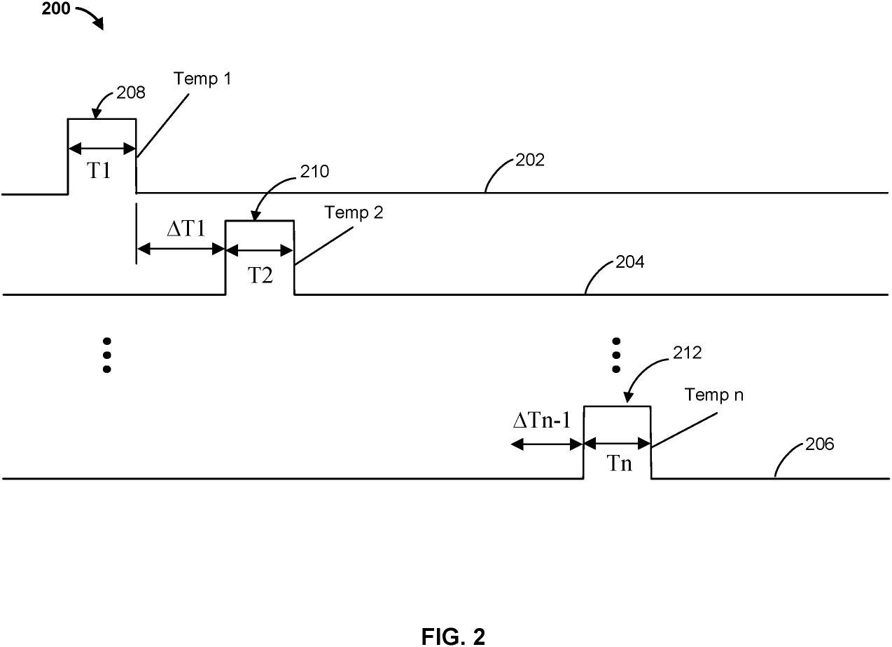

Referring now to FIG. 2, an example heating profile 200 is shown. The profile 200 represents a time at which each of heater 1 through heater n is energized relative to the other heaters. The heating profile 200, in this example, is a sequential profile. As shown, each of the lines 202, 204 and 206 represent when power is delivered to each of heaters 1 through n. As shown, heater 1 (shown at line 202) is energized first and is energized to a sufficient level 208 to raise the temperature of the cryo-fluid to Temp 1. Heater 1 is energized for a period of time T1. Heater 1 is then deenergized and period of time ΔT1 may pass before heater 2 is energize (shown at line 204). Heater 2 is energized to a sufficient level 210 to result in the temperature of the cryo-fluid to be at Temp 2. Heater 2 is energized for a period of time T2 before it is deenergized. As can be appreciated, each of the heaters that may be included in the cryoablation system 100 can be heated in this manner for a number n of heaters. Heater n, as represented by line 206, is energized to a sufficient level 212 to raise the temperature of the cryo-fluid to a temperature Tn.

The power levels 208, 210, 212 are shown to each have substantially the same shapes and the same levels. It should be appreciated, however, that the levels 208, 210, 212 can be different as may be required to adaptively control and raise the temperature of the cryo-fluid to a desired temperature during a thaw cycle. Similarly, the durations or times T1, T2, Tn that each heater is energized may be the same or may be different. In addition, it may be desirable to energize the heaters in order that may skip adjacent heaters. In still further examples, the profiles may include overlapping power profiles in which heaters may be energized at the same time for some periods of time.

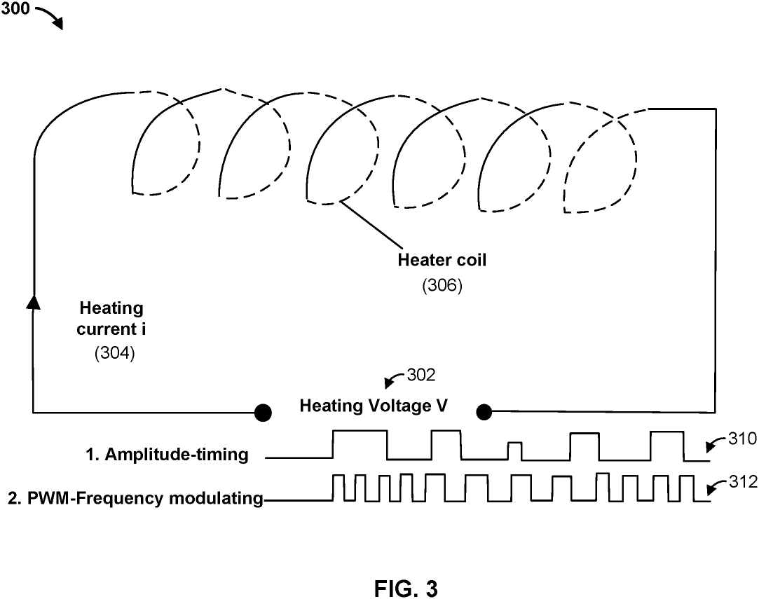

Turning now to FIG. 3, an example heater 300 is shown. In this example, the heater 300 may include a heating coil 306 that is energized with a heating current 304 and a heating voltage 302. The power provided to the heating coil 306 may be varied by the cryoablation computing device 102 and/or the smart multi-heat control 104 by varying the heating current 304 and/or the heating voltage 302. In some examples, the power provided to the heating coil 306 can be substantially continuous during the duration of the heating duration. In other examples, the power can be provided using the methods 310 and/or 312. In one example, the power can be provided in an amplitude-timing method. In the amplitude-timing method 310, the power can be provided such that the amplitude of the power is varied between energizing cycles. The power can also be provided in a pulse-width modulation or frequency modulating method 312. In this method, the duration or frequency of each power cycle can be varied and the amplitude of each cycle may remain substantially the same. In yet other examples, other types of methods of providing power to the heater 300 can be used.

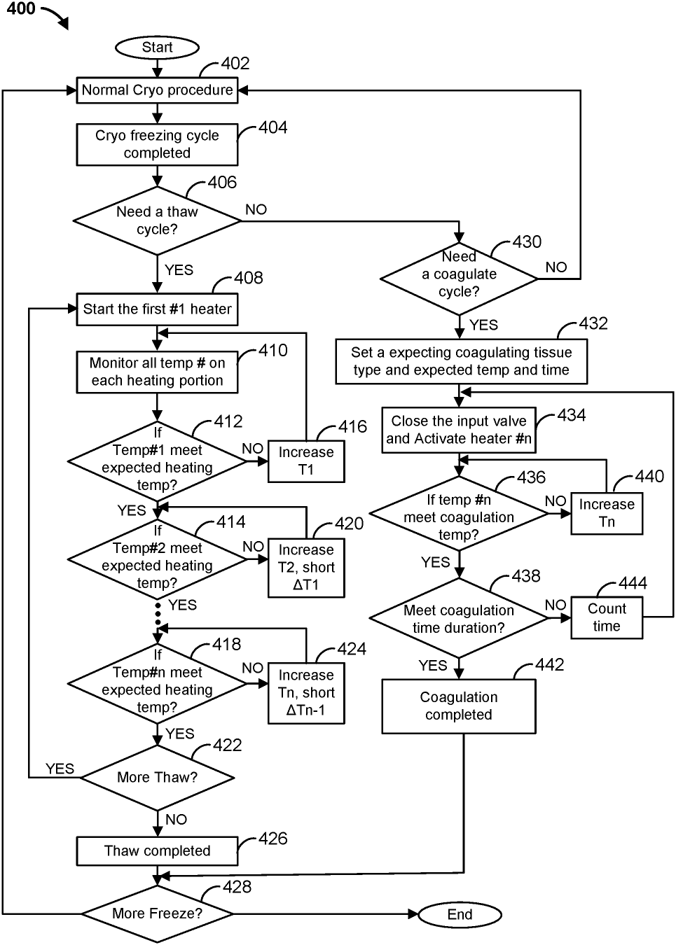

Referring now to FIG. 4, a method 400 of performing a cryoablation procedure is shown. The method 400 may be performed by various cryoablation apparatuses or systems of the present disclosure. For example, the method 400 may be performed by the cryoablation system 100 previously described. The description below describes the method 400 relative to the cryoablation system 100 but it should be appreciated that other systems and apparatuses can also be used.

At step 402, a normal cryo procedure is performed. For the sake of brevity, the freezing cycle of the cryoablation procedure is not repeated. As can be appreciated, the cryoablation system 100 may perform a freezing cycle during which an iceball is formed to destroy a target tissue in a patient.

At step 404, the cryoablation computing device 102 may determine if the cryo freezing cycle is completed. The cryoablation computing device 102 may determine if the freezing cycle is completed by monitoring a temperature and duration of the freezing cycle, for example. In other examples, the cryoablation computing device 102 may make this determination based on other data, signals or other information obtained by the cryoablation computing device 102.

At step 406, the cryoablation computing device 102 may determine if a thaw procedure is required. The cryoablation computing device 102 may, for example, compare a status or historical information to a treatment plan. The treatment plan may, for example, describe the number of freezing and thaw cycles that are to be performed during a treatment procedure. The cryoablation computing device 102 can determine whether a thaw procedure is required based on the treatment plan, for example. In other examples, a user may input whether a thaw cycle is to be performed in response to receiving a message or inquiry from the cryoablation computing device 102. If the cryoablation computing device 102 determines that a thaw cycle is required the method 400 moves to step 408. If the cryoablation computing device 102 determines that a thaw cycle is not required, the method moves to step 430.

At step 408, the cryoablation computing device 102 may initiate the thaw cycle and start the first heater 110. The cryoablation computing device 102 may energize the first heater 110 using any of the methods or profiles previously described, for example.

At step 410, the cryoablation computing device 102 may monitor all the temperatures at all the heating locations on the cryo-fluid supply 124. The cryoablation computing device 102 may monitor the temperature at the first heater 110, at the second heater 112, at the third heater 114, at the fourth heater 116 and at the probe heater 130. Each of the sensors at each location or as provided by the impedance of the heating coil, the smart multi-heat control 104 may receive such temperature information and pass the temperature information to the cryoablation computing device 102.

In order to provide efficient and accurate thawing at the cryoprobe 118, various temperature thresholds may be determined via testing or historical information. The temperature thresholds may describe temperatures of each heating location that are needed in order to achieve a desired thaw at the cryoprobe 118. The temperature thresholds may describe a temperature at the first heater 110, at the second heater 112, at the third heater 114, at the fourth heater 116 and/or at the probe heater 130. The cryoablation computing device 102 may compare the measured temperature at the heating locations with the temperature thresholds.

At step 412, the cryoablation computing device 102 may compare the temperature at the first heater 110 to a first temperature threshold or expected heating temperature. If the temperature at the first location (e.g., at the first heater 110) is at the expected temperature or at the temperature threshold, the method can proceed to step 414. If the cryoablation computing device 102 determines that the temperature at the first location is lower than the temperature threshold, the method can proceed to step 416.

At step 416, the cryoablation computing device 102 may increase the temperature at the first heating location. The cryoablation computing device 102 may increase the temperature by increasing the power delivered to the first heater 110, for example. The cryoablation computing device 102 may also or alternatively increase the time T1 (see FIG. 2) during which the first heater 110 is energized.

At step 414, the cryoablation computing device 102 can compare the temperature at the second heating location to a temperature threshold or expected heating temperature. As can be appreciated, the temperature threshold at the second heating location may be different from the temperature threshold at the first heating location. If the cryoablation computing device 102 determines that the temperature at the second heating location is at least at the temperature threshold, the method can proceed to step 418. If the cryoablation computing device 102 determines that the temperature at the second heating location is less than the temperature threshold, the method may proceed to step 420.

At step 420, the cryoablation computing device 102 may increase temperature at the second heating location. The cryoablation computing device 102 may, for example, increase the power delivered to the second heater 112. The cryoablation computing device 102 may also or alternatively increase the amount of time T2 during which the second heater 112 is energized. As further shown, the cryoablation computing device 102 may also reduce or shorten the time ΔT1 that corresponds to a delay between the energizing of the second heater 112 after the first heater 110.

As shown, the foregoing process of measuring, comparing and then adjusting (if necessary) the heating at each subsequent heating location can be performed in accordance with the amount of heating locations and/or heaters that may be provided in the cryoablation system 100. At step 418, the cryoablation computing device 102 may compare the temperature at heater n to a temperature threshold for heating location n. If the cryoablation computing device 102 determines that the temperature at the heating location n is at or greater than the threshold temperature n, the method can proceed to step 422. If the cryoablation computing device 102 determines that the temperature at the heating location n is less than the temperature threshold for the heating location n, the method proceed to step 424.

At step 424, the cryoablation computing device 102 may increase the temperature at the heating location n. The cryoablation computing device 102 may take action to increase power, increase a duration of the energizing cycle and/or shorten the pause or period of time between subsequent heating by the adjacent heaters.

At step 422, the cryoablation computing device 102 may determine whether the thaw cycle is completed. The cryoablation computing device 102 may determine if the thaw cycle is completed by comparing the duration, profiles, temperatures or other information to the treatment cycle and/or to the temperature thresholds and/or predetermined thaw limits. If the cryoablation computing device 102 determines that the thaw cycle is complete, the method proceed to step 426. If the cryoablation computing device 102 determines that the thaw cycle is not complete, the method returns to step 408 in which the cryoablation computing device 102 continues to monitor and actively adjust the heating profiles and power delivered to each of the heaters in the cryoablation system 100.

At step 426, the cryoablation computing device 102 has determined that the thaw cycle is completed and moves to step 428. At step 428, the cryoablation computing device 102 determines whether another freeze cycle is needed. cryoablation computing device 102 may, for example, determine whether another freeze cycle is completed by comparing the information collected, measured and/or stored by the cryoablation computing device 102 to the treatment plan. The treatment plan may described that two or more freezing cycles are to be performed. If more freeze cycles are to be performed, the method returns to step 402. If no further freeze cycles are needed, the method 400 may end.

Referring back to step 430, the cryoablation computing device 102 may determine that a thaw cycle is not required but may then determine whether a coagulation cycle is needed. A coagulation cycle may be prescribed in a treatment plan or may be determined to be necessary should bleeding occur during the course of treatment. The cryoablation computing device 102 may receive an input from a user that a coagulation cycle is necessary or may determine that a coagulation cycle is necessary by receiving a measurement or other indication that bleeding has occurred. If the cryoablation computing device 102 determines that a coagulation cycle is needed, the method moves to step 432. If the cryoablation computing device 102 determines that a coagulation cycle is not needed, the method can return to step 402.

At step 432, the cryoablation computing device 102 may set various coagulation settings. The settings may include, for example, a tissue type, expected coagulation temperature and a coagulation time (or duration). The settings may be predetermined and obtained by the cryoablation computing device 102 from a database or other repository. In other examples, the settings can be input into a user interface of the cryoablation computing device 102 by a user. The settings can be different for different types of procedures or different types of tissue at which bleeding may occur. For example, the coagulation setting may be different depending on the location of the target tissue. Different coagulation settings are needed, for example, for tissues in the liver than for tissues in a kidney, prostate, lung or other organs or body structures.

At step 434, the cryoablation computing device 102 may close the cryo-fluid valve. The inlet valve 108 can be closed by the cryoablation computing device 102, for example. A heater (denoted as heater n, in the figure) can be activated by the cryoablation computing device 102. The probe heater 130, for example, can be energized. This can cause the cryoprobe 118 to be heated.

At step 436, the cryoablation computing device 102 can compare the temperature at the heating location to a predetermined coagulation temperature. If the cryoablation computing device 102 determines that the temperature at the coagulation location is at or greater than the coagulation temperature, the method may proceed to step 438. If the cryoablation computing device 102 determines that the temperature at the coagulation location is less than the coagulation temperature threshold, the method may proceed to step 440.

At step 440, the time Tn or duration of the coagulation heating can be increased. This increase in time can allow the heater to further warm the area of coagulation.

At step 438, the cryoablation computing device 102 may determine whether the coagulation area has been heated at or above the coagulation temperature threshold for the prescribed coagulation duration. If the area has been heated for the prescribed coagulation time duration, the method may proceed to step 442. If the cryoablation computing device 102 determines that the area has not been heated for the prescribed coagulation time duration, the method returns to step 434 to continue to monitor and adjust the coagulation cycle as necessary.

At step 442, the cryoablation computing device 102 has determined that the coagulation cycle has been completed. The method 400 can return to step 428 as previously described.

The performance of the method 400 can result in improvements over existing or traditional thaw methods in cryoablation procedures. In some examples, the sequential measurement and heating of the heaters in the cryoablation system 100 can significantly improve the ability to control the heating that occurs and to require significantly less power. In one example, the use of method 400 improves the power usage from 20 W in traditional systems to 2 W or less.

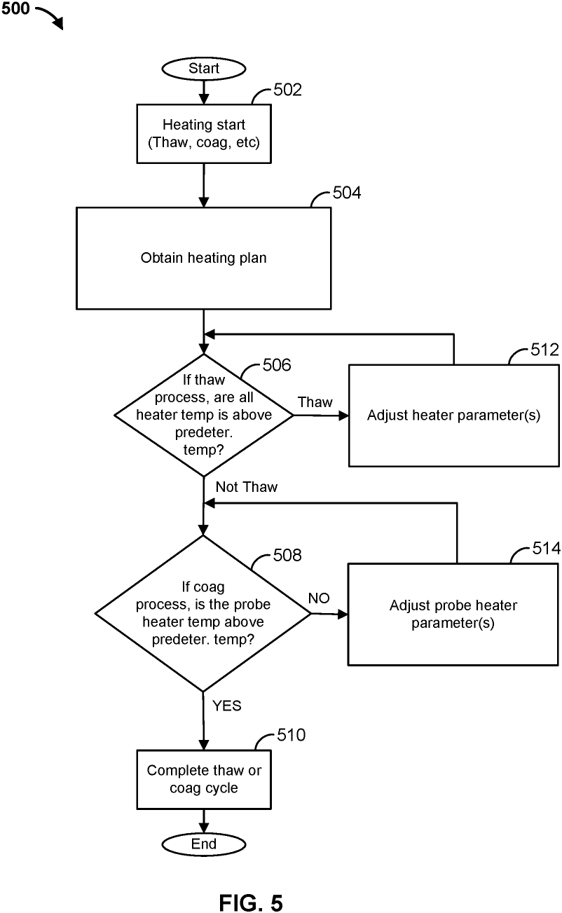

Referring now to FIG. 5, another example method 500 is shown. The method 500 may be performed by various cryoablation apparatuses or systems of the present disclosure. For example, the method 500 may be performed by the cryoablation system 100 previously described. The description below describes the method 500 relative to the cryoablation system 100 but it should be appreciated that other systems and apparatuses can also be used.

At step 502, the heating cycle may begin. The method 500 may be used for a thaw cycle or a coagulation cycle. At step 504, the cryoablation computing device 102 may obtain a heating plan. The heating plan may be stored in a database and retrieved by the cryoablation computing device 102. In other examples, the heating plan may be input by a medical professional or other user. The heating plan may describe a predetermined heating profile that can include types of target tissues, location of target tissue, patient information, temperature thresholds, heating profiles for one or more heaters of the cryoablation system 100, heating temperatures, heating durations, power distribution profiles, power levels and the like.

At step 506, the cryoablation computing device 102 may determine that a thaw cycle is being implemented. In such an instance, the cryoablation computing device 102 can obtain temperature measurements from all the heaters and/or at all the heating locations and compare such measurements and information to the heating plan. In some examples, the cryoablation computing device 102 can determine whether a temperature at each heater or at each heating location is at or above a predetermined temperature threshold as defined in the heating plan. If the temperatures are at or above the predetermined temperature thresholds, the method can proceed to step 510. If the cryoablation computing device 102 determines that the temperatures are below the predetermined temperature thresholds, the method can proceed to step 512.

At step 512, the cryoablation computing device 102 may adjust the heater parameters. The cryoablation computing device 102 may change or adjust the power delivered to the heaters, energize a sequentially positioned heater, vary a time pause between heaters, change a heating duration, change the pulse width, frequency or amplitude of the power delivered to the heater or take other action as may be necessary.

As can be appreciated the loop of steps 506 and 512 may be continuously or periodically performed by the cryoablation computing device 102 during the thaw cycle. The loop may be performed until a time duration is reached or the cryoablation computing device 102 determines that the thawing is complete by measuring a temperature of the cryoprobe 118 for example.

In instance in which a coagulation cycle is prescribed in the treatment plan or when a coagulation cycle is otherwise performed, the method 500 may include the loop with step 508 and step 514. The coagulation loop is similar to the thaw cycle previously described. The coagulation cycle, however, is more concerned with the temperature at the cryoprobe 118 since the cryoprobe is positioned at or adjacent a tissue where bleeding may occur. At step 508, the cryoablation computing device 102 may determine if the temperature at the cryoprobe 118 is at or above the predetermined coagulation temperature. Such temperature may be described in the heating plan or can be determined by the cryoablation computing device 102 based on the location of the cryoprobe 118 or the type of tissue described in the heating plan.

If the cryoablation computing device 102 determines that the cryoprobe temperature is at or above the coagulation temperature threshold, the method can proceed to step 510. If the cryoablation computing device 102 determines that the temperature is below the coagulation temperature threshold, the method can proceed to step 514. At step 514, the cryoablation computing device 102 may adjust one or more parameters of the probe heater 130. The smart multi-heat control 104 may adjust the power, frequency, power profile, pulse width, amplitude or other heating parameter. The loop of steps 508 and 514 can be continuously or periodically performed until the cryoablation computing device 102 determines that the coagulation cycle is complete.

At steps 510, the cryoablation computing device 102 has determined that the thaw or coagulation cycle is complete as described above and the method may end. The active monitoring and controlling of the heaters of the cryoablation system 100 can provide the improvements noted above.

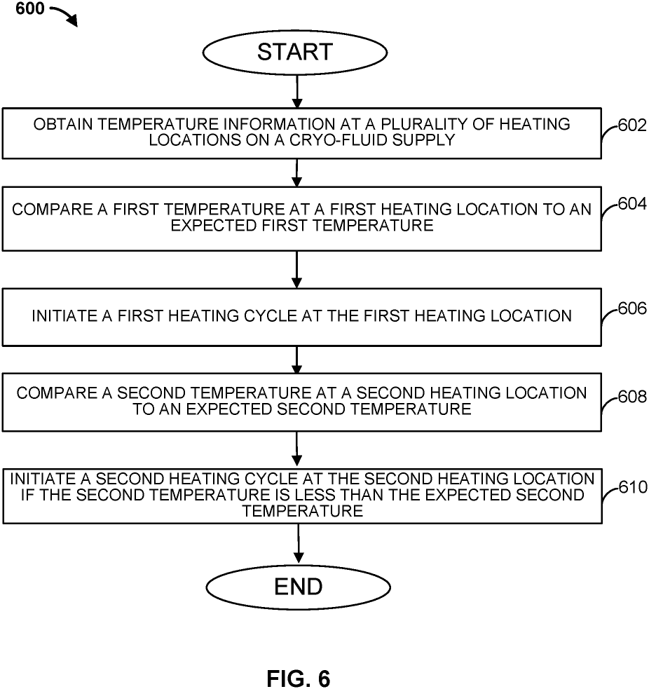

Referring now to FIG. 6, an example method 600 of sequential heating in a cryoablation system is shown. The method 600 may be performed by various cryoablation apparatuses or systems of the present disclosure. For example, the method 600 may be performed by the cryoablation system 100 previously described. The description below describes the method 600 relative to the cryoablation system 100 but it should be appreciated that other systems and apparatuses can also be used.

At step 602, the cryoablation computing device 102 may obtain temperature information for temperatures at one or more heating locations on a cryoablation system. The cryoablation computing device 102 may obtain temperature information for temperatures at each heater on the cryo-fluid supply 124, for example. The cryoablation computing device 102 can obtain temperature measurements at the first heater 110, the second heater 112, the third heater 114, the fourth heater 116 and the probe heater 130. Temperature sensors may be located at each of the heating locations. The heating coils that may be located at each of the heaters may also provide temperature measures via an impedance measurement at each of the coils. The temperature information can be collected and stored by the cryoablation computing device 102.

At step 604, the cryoablation computing device 102 may compare a first temperature at a first heating location to an expected first temperature. For example, the cryoablation computing device 102 may obtain a temperature measurement from the first heater 110. The first heater 110 is located at a position furthest upstream of the cryoprobe 118. The expected first temperature can correspond to a predetermined temperature threshold associated with the first heating location. This information may be obtained by the cryoablation computing device 102 from a treatment plan, heating plan or other predetermined schedule entered, accessed or otherwise obtained by the cryoablation computing device 102.

At step 606, the cryoablation computing device 102 may initiate a first heating cycle at the first heating location. The heating cycle at the first location may be initiated when the cryoablation computing device 102 determines that the temperature at the first heating location is less than the expected first temperature. The heating cycle may include any suitable heating such as providing a power level, power profile, amplitude-timing power signal, PWM power signal or the like. The cryoablation computing device 102 may determine a recommended power profile based on a treatment plan or other inputs regarding the cryoablation treatment including tissue, location, patient health and the like.

At step 608, the cryoablation computing device 102 may compare a second temperature at a second heating location to an expected second temperature. Step 608 is similar in many respects to step 604. The second heating location, however, may be located downstream of the first heating location. In this manner, the cryo-fluid may be heated in a sequential manner by heaters positioned along the cryo-fluid supply 124 in a direction downstream of the cryo-fluid source 106.

At step 610, the cryoablation computing device 102 may initiate a second heating cycle at the second heating. The cryoablation computing device 102 may initiate the second heating cycle if it determines that the second temperature is less than the expected second temperature.

The method 600, while not shown, may include further steps of comparing temperatures to expected temperatures at further heating locations located sequentially downstream of the first heating location and the second heating location. As can be appreciated similar steps can be included in the method 600 for the third heater 114, the fourth heater 116 and/or the probe heater 130 of the cryoablation system 100.

The cryoablation computing device 102 may initiate heating cycles by energizing the heaters of the cryoablation system 100 using the same or different profiles for each heater. The power profiles may include amplitude modulated power profiles and/or pulse width modulated power profiles.

Referring now to FIG. 7, an example computing device 700 is shown. The cryoablation system 100 may include one or more computing devices 700. For example, the cryoablation computing device 102 may have the elements shown in FIG. 7. The methods of the present disclosure, such as methods 400, 500, and 600, may be performed, or steps of such methods may be performed, by a computing device 700.

As shown, the computing device 700 may include one or more processors 702, working memory 704, one or more input/output devices 706, instruction memory 708, a transceiver 712, one or more communication ports 714, and a display 716, all operatively coupled to one or more data buses 710. Data buses 710 allow for communication among the various devices. Data buses 710 can include wired, or wireless, communication channels.

Processors 702 can include one or more distinct processors, each having one or more cores. Each of the distinct processors can have the same or different structure. Processors 702 can include one or more central processing units (CPUs), one or more graphics processing units (GPUs), application specific integrated circuits (ASICs), digital signal processors (DSPs), and the like.

Processors 702 can be configured to perform a certain function or operation by executing code, stored on instruction memory 708, embodying the function or operation. For example, processors 702 can be configured to perform one or more of any function, step, method, or operation disclosed herein.

Instruction memory 708 can store instructions that can be accessed (e.g., read) and executed by processors 702. For example, instruction memory 708 can be a non-transitory, computer-readable storage medium such as a read-only memory (ROM), an electrically erasable programmable read-only memory (EEPROM), flash memory, a removable disk, CD-ROM, any non-volatile memory, or any other suitable memory.

Processors 702 can store data to, and read data from, working memory 704. For example, processors 702 can store a working set of instructions to working memory 704, such as instructions loaded from instruction memory 708. Processors 702 can also use working memory 704 to store dynamic data created during the operation of cryoablation computing device 102. Working memory 704 can be a random access memory (RAM) such as a static random access memory (SRAM) or dynamic random access memory (DRAM), or any other suitable memory.

Input-output devices 706 can include any suitable device that allows for data input or output. For example, input-output devices 706 can include one or more of a keyboard, a touchpad, a mouse, a stylus, a touchscreen, a physical button, a speaker, a microphone, or any other suitable input or output device.

Communication port(s) 714 can include, for example, a serial port such as a universal asynchronous receiver/transmitter (UART) connection, a Universal Serial Bus (USB) connection, or any other suitable communication port or connection. In some examples, communication port(s) 714 allows for the programming of executable instructions in instruction memory 708. In some examples, communication port(s) 714 allow for the transfer (e.g., uploading or downloading) of data.

Display 716 can display a user interface 718. User interfaces 718 can enable user interaction with the cryoablation computing device 102. For example, user interface 718 can be a user interface that allows an operator to interact, communicate, control and/or modify different messages, settings, or features that may be presented or otherwise displayed to a user. The user interface 718 can include a slider bar, dialogue box, or other input field that allows the user to control, communicate or modify a setting, limitation or input that is used in a cryoablation treatment. In addition, the user interface 718 can include one or more input fields or controls that allow a user to modify or control optional features or customizable aspects of the cryoablation computing device 102 and/or the operating parameters of the cryoablation system 100. In some examples, a user can interact with user interface 718 by engaging input-output devices 706. In some examples, display 716 can be a touchscreen, where user interface 718 is displayed on the touchscreen. In other examples, display 716 can be a computer display that can be interacted with using a mouse or keyboard.

Transceiver 712 allows for communication with a network. In some examples, transceiver 712 is selected based on the type of communication network cryoablation computing device 102 will be operating in. Processor(s) 702 is operable to receive data from, or send data to, a network, such as wired or wireless network that couples the elements of the cryoablation system 100 of FIG. 1.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system for performing a cryoablation treatment, the system comprising at least one computing device configured to:
obtain temperature information at a plurality of heating locations on a cryo-fluid supply, the plurality of heating locations comprising a first heating location and a second heating location;
compare a first temperature at the first heating location to an expected first temperature;
intiate a first heating cycle at the first heating location if the first temperature at the first heating location is less than the expected first temperature;
compare a second temperature at the second heating location to an expected second temperature, the second heating location disposed downstream of the first heating location; and
initiate a second heating cycle at the second heating location if the second temperature is less than the expected second temperature.

2. The system of claim 1, further comprising one or more temperature sensors coupled to the at least one computing device, wherein the at least one computing device obtains the temperature information from one or more temperature sensors.

3. The system of claim 1, further comprising a plurality of heating coils each positioned at a corresponding heating location of the plurality of heating locations.

4. The system of claim 3, wherein each heating coil is configured to send a temperature signal to the at least one computing device, the temperature signal corresponding to a temperature at the corresponding location on the cryo-fluid supply.

5. The system of claim 4, wherein each heating coil is also configured to selectively heat a cryo-fluid in the cryo-fluid supply at the corresponding heating location.

6. The system of claim 1, wherein the computing device is configured to energize a first heater at the first heating location to initiate the first heating cycle.

7. The system of claim 1, wherein the at least one computing device if further configured to:
compare a third temperature at a third heating location to an expected third temperature, the third heating location disposed downstream of the first heating location and the second heating location; and
initiate a third heating cycle at the third heating location if the third temperature is less than the expected third temperature.

8. The system of claim 1, wherein the second heating location is disposed upstream of a cryoprobe.

9. The system of claim 1, wherein the first heating cycle comprises a first temperature profile and the second heating cycle comprises a second heating profile, the first temperature profile and the second temperature profile being different.

10. The system of claim 1, wherein the first heating cycle comprises an amplitude modulated power profile.

11. The system of claim 1, wherein the first heating cycle comprises a pulse width modulated (PWM) power profile.

12. A method of sequentially heating for a cryoablation system, the method comprising:
obtaining temperature information at a plurality of heating locations on a cryo-fluid supply, the plurality of heating locations comprising a first heating location and a second heating location;
comparing a first temperature at the first heating location to an expected first temperature;
intiating a first heating cycle at the first heating location if the first temperature at the first heating location is less than the expected first temperature;
comparing a second temperature at the second heating location to an expected second temperature, the second heating location disposed downstream of the first heating location; and
initiating a second heating cycle at the second heating location if the second temperature is less than the expected second temperature.

13. The method of claim 12, wherein the at least one computing device obtains the temperature information from one or more temperature sensors.

14. The method of claim 12, wherein the first heating coil is initiated by energizing a first heater coil and the temperature information is at least partially obtained from the first heater coil.

15. The method of claim 14, wherein the first heating coil is configured to send a temperature signal to at least one computing device, the temperature signal corresponding to a temperature at the corresponding location on the cryo-fluid supply.

16. The method of claim 15, wherein the first heating coil s also configured to selectively heat a cryo-fluid in the cryo-fluid supply at the corresponding heating location.

17. The method of claim 12, wherein at least one computing device is configured to energize a first heater at the first heating location to initiate the first heating cycle.

18. The method of claim 12, further comprising:
comparing a third temperature at a third heating location to an expected third temperature, the third heating location disposed downstream of the first heating location and the second heating location; and
initiating a third heating cycle at the third heating location if the third temperature is less than the expected third temperature.

19. The method of claim 12, wherein the second heating location is disposed upstream of a cryoprobe.

20. The method of claim 12, wherein the first heating cycle comprises a first temperature profile and the second heating cycle comprises a second heating profile, the first temperature profile and the second temperature profile being different.
